(19) United States
(12) Patent Application Publication
(10) Pub. No.: US 2025/0125020 A1
(43) Pub. Date: Apr. 17, 2025

(54) METHOD AND DEVICE, ELECTRONIC EQUIPMENT AND STORAGE MEDIUM FOR TRAINING AND OPTIMIZING ANALYSIS MODEL

(71) Applicants: Douyin Vision Co., Ltd., Beijing (CN); Lemon Inc., Grand Cayman (KY)

(72) Inventors: Weihao GAO, Beijing (CN); Sheng GONG, Los Angeles, CA (US); Zhenliang MU, Beijing (CN); Yumin ZHANG, Los Angeles, CA (US)

(21) Appl. No.: 18/896,056

(22) Filed: Sep. 25, 2024

(30) Foreign Application Priority Data
Oct. 12, 2023 (CN) 202311322469.2

(51) Int. Cl. G16C 20/70; G16C 20/30; G16C 60/00

(52) U.S. Cl. CPC G16C 20/70; G16C 20/30; G16C 60/00

(57) ABSTRACT

The embodiment of the invention provides method, apparatus, device and a storage medium for training and optimizing an analysis model. The method of optimizing the analysis model includes: fine-tuning an analysis model with a first set of values regarding a first property of a target material to determine a second set of values regarding a second property of the target material; determining an association between the first property and the second property of the target material based on a first set of values and a second set of values; determining a target value of the target material regarding the first property with the association based on a reference value of the target material regarding the second property, the reference value being determined based on an experiment on target material; and optimizing the analysis model with the target value of the target material regarding the first property. In this way, embodiments of the present disclosure can utilize limited experimental data to optimize the analysis model.

### CROSS-REFERENCE TO RELATED APPLICATIONS

This application claims priority to Chinese Patent Application No. CN202311322469.2, filed on Oct. 12, 2023, and entitled “METHOD AND DEVICE, ELECTRONIC EQUIPMENT AND STORAGE MEDIUM FOR TRAINING AND OPTIMIZING ANALYSIS MODEL”, the entirety of which is incorporated here by reference.

### FIELD

Example embodiments of the present disclosure generally relate to the field of computers, and in particular, to a method and apparatus for training and optimizing an analysis model, an electronic device, and a computer-readable storage medium.

### BACKGROUND

Electrolytes play an indispensable role in wearable devices, electric vehicles, and static energy storage devices. In addition, electrolyte research has important significance in non-energy storage applications, such as in food, environmental and pharmaceutical fields.

With the advancement of computing power, the design and iteration of electrolyte gradually presents a digital trend. However, current tools cannot simultaneously meet the authenticity and accuracy of electrolyte simulation.

### SUMMARY

In a first aspect of the present disclosure, a method for optimizing an analysis model is provided. The method comprises: fine-tuning an analysis model with a first set of values regarding a first property of a target material to determine a second set of values regarding a second property of the target material; determining an association between the first property and the second property of the target material based on a first set of values and a second set of values; determining a target value of the target material regarding the first property with the association based on a reference value of the target material regarding the second property, the reference value being determined based on an experiment on target material; and optimizing the analysis model with the target value of the target material regarding the first property.

In a second aspect of the present disclosure, a method for training an analysis model is provided. The method comprises: determining a set of feature representations of a set of atoms in an electrolyte sample using an analysis model; determining electric charge information of the electrolyte sample using the analysis model based on the set of feature representations; determining energy information of the electrolyte sample with the analysis model based on the electric charge information and the set of feature representations; and training the analysis model based on the electric charge information and the energy information, wherein a loss function for training the analysis model comprises at least a first part and/or a second part, the first part being associated with a multipolar moment determined based on the electric charge information, the second part being associated with force information determined based on the energy information.

In a third aspect of the present disclosure, a method for training an analysis model is provided. The method comprises: obtaining a plurality of analysis models for analyzing a target material, a plurality of analysis models being trained based on different initial values of model parameters; determining a plurality of prediction results regarding forces of a set of atoms in the target material using a plurality of analysis models; determining target data for forces of a set of atoms based on the plurality of prediction results; and training, using the target data, a target analysis model for analyzing the target material.

In a fourth aspect of the present disclosure, an apparatus for optimizing an analysis model is provided. The apparatus comprises: a fine-tuning model configured to fine-tune an analysis model with a first set of values regarding a first property of a target material to determine a second set of values regarding a second property of the target material; a first determination model configured to determine an association between the first property and the second property of the target material based on a first set of values and a second set of values; a second determination module configured to determine a target value of the target material regarding the first property with the association based on a reference value of the target material regarding the second property, the reference value being determined based on an experiment on target material; and a optimization module configured to optimize the analysis model with the target value of the target material regarding the first property.

In a fifth aspect of the present disclosure, an apparatus for training an analysis model is

provided. The apparatus comprises: a feature determination module configured to determine a set of feature representations of a set of atoms in an electrolyte sample using an analysis model; an electric charge determination module configured to determine electric charge information of the electrolyte sample using the analysis model based on the set of feature representations; an energy determination module configured to determine energy information of the electrolyte sample with the analysis model based on the electric charge information and the set of feature representations; and a first training module configured to train the analysis model based on the electric charge information and the energy information, wherein a loss function for training the analysis model comprises at least a first part and/or a second part, the first part being associated with a multipolar moment determined based on the electric charge information, the second part being associated with force information determined based on the energy information.

In a sixth aspect of the present disclosure, an apparatus for training an analysis model is provided. The apparatus comprises: a model obtaining module configured to obtain a plurality of analysis models for analyzing a target material, a plurality of analysis models being trained based on different initial values of model parameters; a result prediction module configured to determine a plurality of prediction results regarding forces of a set of atoms in the target material using a plurality of analysis models; a third determination module configured to determine target data for forces of a set of atoms based on the plurality of prediction results; and second training module configured to train, using the target data, a target analysis model for analyzing the target material.

In a seventh aspect of the present disclosure, an electronic device is provided. The apparatus includes at least one processing unit; and at least one memory coupled to the at least one processing unit and storing instructions for execution by the at least one processing unit. The instructions, when executed by the at least one processing unit, cause the electronic device to perform the method of the first aspect, the second aspect, or the third aspect.

In an eighth aspect of the present disclosure, a computer-readable storage medium is provided. The medium stores a computer program, and when the computer program is executed by the processor, the method of the first aspect, the second aspect, or the third aspect is implemented.

It should be understood that the content described in this section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent from the following detailed description taken in conjunction with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2 shows a flowchart of an example process of training an analysis model according to some embodiments of the present disclosure;

FIG. 3 illustrates an example architecture of an analysis model according to some embodiments of the present disclosure;

FIG. 4 shows a flowchart of an example process of optimizing an analysis model according to some embodiments of the present disclosure;

FIG. 5 illustrates a schematic diagram of fine-tuning analysis model according to some embodiments of the present disclosure;

FIG. 6 is a flowchart of an example process of training an analysis model according to some other embodiments of the present disclosure;

FIGS. 7A-7I illustrate a model performance comparison diagram according to some embodiments of the present disclosure;

FIG. 8A shows a schematic structural block diagram of an apparatus for optimizing an analysis model according to some embodiments of the present disclosure;

FIG. 8B is a schematic structural block diagram of an apparatus for training an analysis model according to some embodiments of the present disclosure;

FIG. 8C shows a schematic structural block diagram of an apparatus for training an analysis model according to some embodiments of the present disclosure; and

FIG. 9 illustrates a block diagram of an electronic device capable of implementing various embodiments of the present disclosure.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for exemplary purposes only and are not intended to limit the scope of the present disclosure.

It should be noted that the headline of any section/subsection provided herein is not limiting. Various embodiments are described throughout and any type of embodiments may be included in any section/subsection. Furthermore, the embodiments described in any section/subsection may be combined in any manner with the same section/subsection and/or any other embodiment described in different sections/subsections.

In the description of the embodiments of the present disclosure, the terms “including” and the like should be understood to include “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below. The terms “first,” “second,” and the like may refer to different or identical object. Other explicit and implicit definition may also be included below.

Embodiments of the present disclosure may relate to data of a user, acquisition and/or use of data, and the like. These aspects all follow the corresponding laws and regulations and related regulations. In the embodiments of the present disclosure, all data is collected, obtained, processed, processed, forwarded, used, etc., all of which are performed on the premise that the user knows and confirms. Accordingly, when implementing the embodiments of the present disclosure, the types of the data or information that may be involved, the usage scope, the usage scenario, and the like should be notified to the user and obtain the authorization of the user in an appropriate manner according to the relevant laws and regulations. The specific notification and/or authorization manner may vary according to actual situations and application scenarios, and the scope of the present disclosure is not limited in this respect.

According to the solutions in the present specification and the embodiments, for example, personal information processing is involved, processing may be performed on the premise of having a legality basis (for example, obtaining consent of a personal information subject, or necessary for performing a fulfillment contract), and processing only within a specified or agreed range. The user rejects personal information other than necessary information required by the basic function, and does not affect the basic function of the user.

As used herein, the term “solution” refers to a system formed by mixing two or more materials. For example, the solution may include a matrix and a solute salt added to the matrix. In embodiments of the present disclosure, the solution may have any suitable matter morphology including, but not limited to, liquid, solid, gaseous, and the like. The electrolyte is formed by adding a metal salt to the solvent. The electrolyte is one example of a solution.

As briefly mentioned above, with the advancement of computing power, the design and iteration of electrolyte gradually presents a digitized trend. However, current tools cannot simultaneously meet the authenticity and accuracy of electrolyte simulation.

High precision and first principles based methods, such as Density Functional Theory (DFT) and AIMD (Ab Initio Molecular Dynamic) simulation are only applicable to simulations on the scale of nanometers and nanosecond, and cannot meet the effective prediction of key properties such as density, viscosity, dielectric constant and ion conductivity of electrolyte materials in high throughput. Although the simulation method based on empirical vertical field molecular dynamics (Molecular Dynamics, MD) can encompass both micron-scale and microsecond scale, since the precision is too low, and the MD is used to describe the energy curve fitting of intramolecular and intermolecular interactions depending on a preset parameter form, its extrapolation ability and use range are limited, and can only be marginal for preliminary exploration of material qualitative.

In addition to the previously mentioned material simulation method, with the continuous progress of artificial intelligence technology, the method of fusing artificial intelligence technology (AI) and material simulation also gradually becomes the emergence angle. This fusion brings the concept of Machine Learning Force Fields (MLFF). The MLFF is intended to achieve a range similar to the molecular dynamics (MD) simulation on a simulated scale, and is calculated in close proximity to the first principle, so that reliable and experimental results consistent with experimental results are predicted.

In training the MLFF, training data needs to be generated first through the AIMD and the DFT. The key step is to select an appropriate and reliable generic function to describe the electron density of the system. However, selection of functional groups usually has certain experience, which results in different material prediction results given by MLFs trained by different functional functions. It is often necessary to spend a lot of time and human resources for finding the applicable functional by means of constant trial and error, and has no mobility, for example, the functional suitable for liquid/polymer is not necessarily suitable for inorganic amorphous.

In addition, most machine learning model frameworks assume that potential energy surfaces can be represented by the summation of a series of atoms within a certain short range fixed truncation distance, often ignoring or underestimating long-range electrostatic interaction forces to some extent. This results in challenging properties such as density, viscosity, dielectric constant, ion conductance, and the like that are affected by accurately capturing long-range electrostatic interaction forces in liquid and other amorphous contexts. The above two limitations make the machine learning model of the part of the sensor have a disregarded gap from the experiment on the prediction of the property of the amorphous electrolyte material. The above two limitations make most of the machine learning model have a disregarded gap from experiments on the prediction of the properties prediction the amorphous electrolyte material.

Various example implementations of this solution are described in detail below in conjunction with the accompanying drawings.

### Example Environment

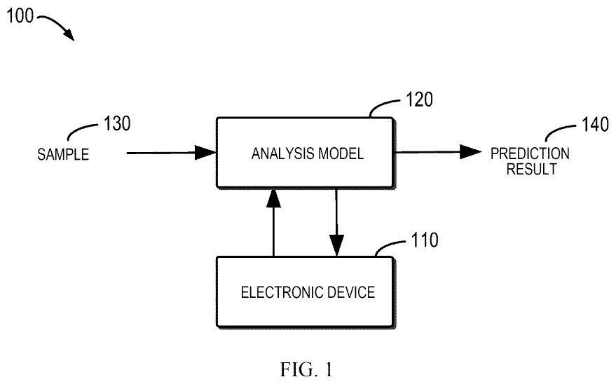

FIG. 1 illustrates a schematic diagram of an example environment 100 in which embodiments of the present disclosure can be implemented. As shown in FIG. 1, the example environment 100 may include an electronic device 110.

In this example environment 100, the electronic device 110 may be used to train and optimize the analysis model 120. The analysis model 120 may be used to predict properties of the material, such as density, viscosity, conductivity, and the like. Therefore, the analysis model 120 may also be referred to as a property prediction model. For example, such an analysis model 120 may be used in the simulation of the electrolyte to prediction the properties of the electrolyte. As will be described in detail below, the analysis model 120 may, for example, receive a sample 130 (e.g., electrolyte or solution) to be simulated and generate a prediction result 140 regarding a particular property of the sample 130.

The training and optimization process of the analysis model 120 will be described in detail below.

### Joint Training of Analysis Model

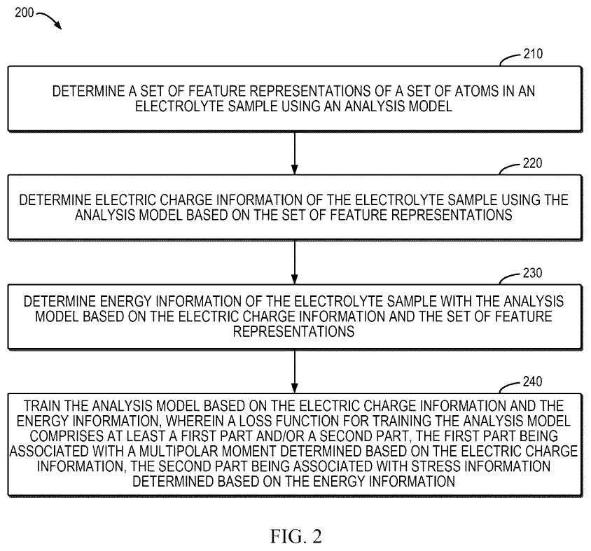

FIG. 2 illustrates a flowchart of an example process 200 of constructing a ligand generative model according to some embodiments of the present disclosure. Process 200 may be implemented at electronic device 110. The process 200 is described below with reference to FIG. 1.

At block 210, the electronic device 110 determines a set of feature representations of a set of atoms in an Electrolyte sample using an analysis model.

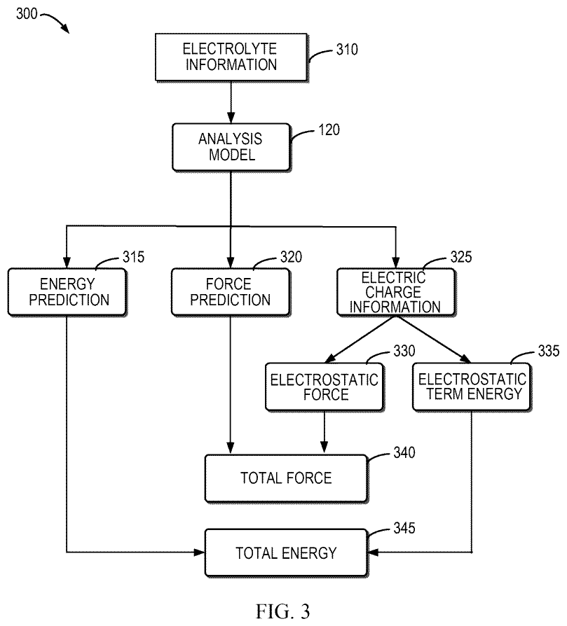

The process 200 will be described below with reference to FIG. 3, which illustrates an example architecture according to some embodiments of the present disclosure. As shown in FIG. 3, the analysis model 120 may receive electrolyte information 310 for an electrolyte sample.

In some embodiments, such electrolyte information 310 may include, for example, atomic positions, atomic types, and side information.

In some embodiments, to ensure rotational translation invariance/invariance in the MLFF process, the analysis model 120 may, for example, utilize Graph Neural Networks (GNNs) to determine the characteristic representation of each atom in the electrolyte.

Specifically, the analysis model 120 may determine a graph representation corresponding to a set of atoms in the electrolyte sample, where a plurality of nodes in the graph representation correspond to a set of atoms, and an edge in the graph representation indicates that a distance between the corresponding two atoms is less than a second threshold.

Further, the analysis model 120 may determine a set of feature representations of a set of atoms in the electrolyte sample by processing the graph representation with a graph neural network of the analysis model.

Thus, GNN can model each atom in the system as a node in the graph, and a pair of atoms whose distance is less than a certain threshold is connected to one edge to form a graph. Multiple rounds of information transfer are performed between the atoms to update the features on the atoms. Finally, the energy, force, and other information of the system are predicted by features on and on the atoms. Strict rotational translation invariance/invariance can be achieved in the graph neural network.

The GNN processing process may update the feature representation of each atom through an attention mechanism. However, in the case of global attention, its computational complexity will reach O (N²). In the electrolyte simulation, the common scenario is a 10000 atom system, so the complexity of O (N²) will cause a very slow simulation.

In some embodiments, the analysis model 120 may employ a local attention mechanism to update the feature representation of the atom. Specifically, the analysis model 120 may determine a first feature representation for a first atom of a set of atoms, and may further determine, based on a distance from the set of atoms to the first atom, at least one second atom whose distance to the first atom is less than a third threshold. Exemplarily, the analysis model 120 may consider only one atom closest to the first atom.

Additionally, the analysis model 120 may update the first feature representation of the first atom with the feature representation of the at least one second atom. Further, the analysis model 120 may determine a set of feature representations of the set of atoms based on the updated first feature representation.

Thus, in the feature transfer process, the attention mechanism may only function between atoms connected with edges. With this method, the complexity will be reduced to O (N), thereby greatly improving the simulation efficiency.

At block 220, the electronic device 110 determines electric Charge Information of the Electrolyte sample using the analysis model based on the set of feature representations.

Specifically, after determining the feature representation (i.e., embedding) of each atom, the electronic device 110 may utilize a multilayer perceptron (MLP, Multilayer Perceptron) included in the analysis model 120 to determine the electric charge information 325 of each atom.

In some embodiments, the analysis model 120 may further ensure that the charge is bounded by one Tanh layer, and ensure that the total charge of the system is constant by subtracting prediction predicted mean value.

At block 230, the electronic device 110 determines energy information of the Electrolyte sample with the analysis model based on the electric Charge Information and the set of feature representations.

Specifically, the feature representation of each atom and prediction predicted charge information 325 may pass through a multilayer perceptron (MLP) in the analysis model 120 to determine the energy of each atom. Further, energy information, that is, energy prediction 315, may be obtained by adding and obtaining energy information, which may be expressed as:

$$E = {\sum }_{i} {E}_{MLP} ( {emb}_{i} ) \qquad ( 1 )$$

where, embᵢ represents a feature representation of the i^{th} atom.

at block 240, the electronic device 110 trains the analysis model based on the electric charge information and the energy information, wherein a loss function for training the analysis model comprises at least a first part and/or a second part, the first part being associated with a multipolar moment determined based on the electric charge information, the second part being associated with force information determined based on the energy information.

Specifically, the electronic device 110 may train the analysis model 120 by jointly training a plurality of loss functions. In some embodiments, the electronic device 110 may determine a multipole moment of the system based on the electric charge information 325, such a multipole moment may correspond to a multi-level expanded 2 or higher order term. Exemplarily, such a multi-pole moment may include a quadrupole moment, an eight-pole moment, etc. of the system.

Exemplarily, the electronic device 110 may use a mean square error (MSE) of the multipole moment of the system as part of a loss function of the model training to perform model training.

In physics, a multipole deployment approach is applied to the approximation of the potential across the charge distribution. The charge and dipole moments respectively correspond to 0-order terms and 1-order terms of multipole expansion. The introduction of the multipole moment as the training target helps the model better prediction the high-order term of multipole expansion, so that the potential is better approximated.

Additionally, the loss function further includes a third portion associated with the electric charge information and/or the dipole moment determined based on the electric charge information. For example, the electronic device 110 may jointly train the electric charge information, the dipole moment, and the mean square error of the multi-pole moment as a training loss function of the model.

In some embodiments, the electronic device 110 may also determine force information, e.g., a virial tensor, of the system based on the energy prediction 315, which may be determined, for example, based on the following process:

$${F}_{i} = - {∇}_{{R}_{i}} E \qquad ( 2 )$$

$$V = {\sum }_{i} {F}_{i} \times {R}_{i} \qquad ( 3 )$$

wherein, as shown in formula (2), the negative derivative of the total energy of the system for each atomic position is the atomic force; as shown in formula (3), the dimension tensor of the system is defined as the sum of the atomic force and the outer product of the atomic position.

The pressure calculation of the system is based on the diagonal terms of the dimension tensor, and the calculation of the viscosity is based on the non-diagonal terms, so the dimension tensor of the better prediction system is favorable for better prediction system properties.

In addition, considering that GNN is a map constructed based on nearest neighbors, it cannot depict interatomic long-range interactions (i.e., electrostatic interactions and dispersion effects). As shown in FIG. 3, the total energy 345 predicted by the analysis model 120 may be based on a combination of energy predicted by the neural network (i.e., energy prediction 315), electrostatic energy 335, and dispersion energy (not shown in FIG. 3). The electrostatic term energy 335 may be obtained, for example, based on a coulomb formula, while the dispersion energy is determined by dispersion correction of the DFT.

Accordingly, the total force 340 may also be a combination of forces (i.e., force predictions 320), electrostatic forces 330, and dispersion forces (not shown in FIG. 3) prediction based on that network. In some number, in order to prevent the electrostatic force and the dispersion force from being too large when the atomic distance is too close, a damping function may be introduced to ensure that the two long-haul functions are attenuated to 0 in a short range.

Therefore, the electronic device 110 may jointly train the analysis model by using a loss function of multiple dimensions. In some embodiments, the value of the loss function is determined based on a comparison with reference data for the electrolyte sample, the reference data being generated based on a density functional theory DFT.

### Optimization of Analysis Model

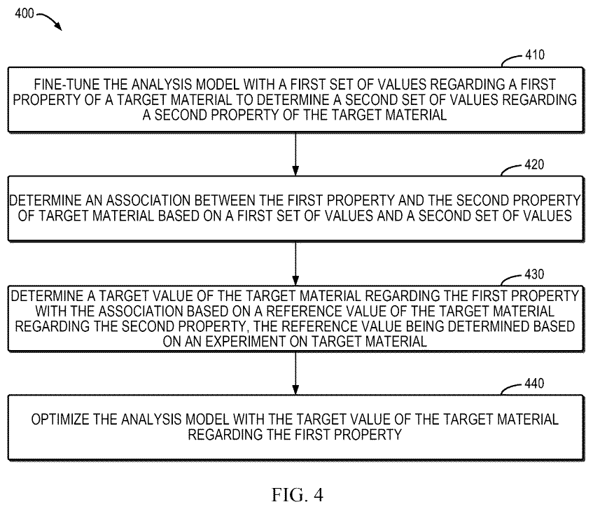

In the machine learning field, density functional theory (DFT) is usually used to generate data, in the simulation of the electrolyte, the number of atoms of the system is usually more than 10000, and the DFT cannot calculate the system of tens of thousands of atoms due to its high time complexity. Therefore, it is common practice to intercept a smaller cluster (usually about 100 atom) from the simulated system, and calculate by using DFT, which in turn causes another problem, that is, on a smaller cluster, the magnitude of the intermolecular force is much smaller than the intramolecular force, so that the MLFF focuses on the acting force in the learning molecule, and the inaccuracy of the intermolecular force is not accurate enough, resulting in inaccurate estimation in a large system. The process of optimizing the analysis model by using limited experimental data according to an embodiment of the present disclosure will be described below with reference to FIG. 4.

FIG. 4 illustrates a flowchart of an example process 400 of optimizing an analysis model according to some embodiments of the present disclosure. Process 400 may be implemented at electronic device 110. The process 400 is described below with reference to FIG. 1.

At block 410, the electronic device 110 fine-tunes the analysis model with a first set of values regarding a first property of a target material to determine a second set of values regarding a second property of the target material.

In some embodiments, the target material may include, for example, a solution. In some cases, the experimental data of the solution is relatively small compared to experimental data of pure solvents. Experiments show that under the condition that a small amount of pure solvent experimental data is used, the prediction of a multi-species and multi-proportion solution can be extrapolated.

In some embodiments, the first property and the second property may be properties of the target material having a predetermined association relationship, wherein the first property is property that can be predicted by the MLFF, the second property is relatively easy to obtain, and measurement accuracy is high.

The first property is the pressure of the solution, and the second property is the density. It should be understood that such first and second properties may also include other suitable examples, for example, the first property may be the dipole moment of the solution, and the second property may be the conductivity of the solution.

There is a linear relationship for the density and pressure of the liquid. As an example, such a linear relationship may be represented by the following equation:

$$β = \frac{1}{ρ} \frac{d ρ}{dP} = \frac{d ln ρ}{dP} \qquad ( 4 )$$

where β represents the compressibility of the liquid, ρ represents the density, and P represents the pressure. Thus, for solution, it can be known that its correct density is ρ₀.

Thus, the electronic device 110 may first utilize the unoptimized analysis model and determine the density ρ₁ of the first round based on the NPT system, and the corresponding pressure may be, for example, P₁. P₁ may be, for example, a preset pressure value, that is, a preset value about the first property (pressure).

Further, the electronic device 110 may determine a first set of values for the first property. For example, the electronic device 110 may determine the first set of values based on a preset value (e.g., P₁) and at least one variation (e.g., at least one dP) for the first property (i.e., pressure).

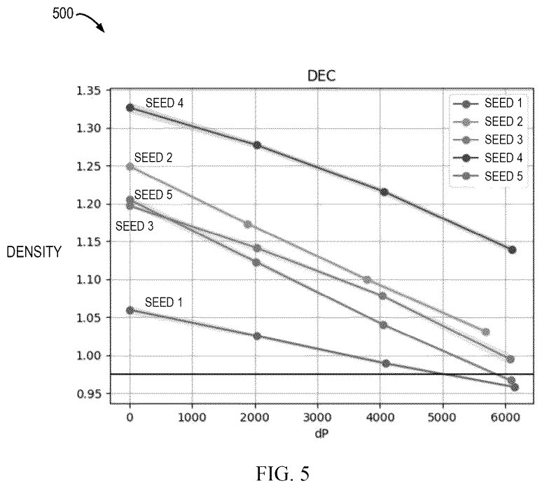

FIG. 5 shows a schematic diagram 500 of fine-tuning analysis model according to some embodiments of the present disclosure. As shown in FIG. 5, the plurality of dPs may be: 2000, 4000, and 6000. Accordingly, the first set of values may be represented as P₁+2000, P₁+4000, and P₁+6000.

Accordingly, the electronic device 110 may fine-tune the model with the determined first set of values for the first property (P₁+2000, P₁+4000 and P₁+6000), so that a set of fine-tuned intermediate model may be determined.

Further, the electronic device 110 may utilize a set of intermediate models to determine a second set of values for the target material regarding the second property. For example, the electronic device 110 may use the trimmed MLFF and determine the density of the second round according to the NPT system, e.g., ρ₂.

At block 420, the electronic device 110 determines an association between the first property and the second property of target material based on a first set of values and a second set of values.

Using the density and pressure as an example, the electronic device 110 may linearly fitting the first set of values and the second set of values to determine an association between the first property and the second property of the target material. It should be understood that, in a case where the properties are different, there may be different manners of determining the relevance. Taking FIG. 5 as an example, the electronic device 110 may linearly fitting dP and d(ln ρ).

At block 430, the electronic device 110 determines a target value of the target material regarding the first property with the association based on a reference value of the target material regarding the second property, the reference value being determined based on an experiment on target material.

Further, after determining the association, the electronic device 110 may determine the target value of the pressure, or the target dP, based on the reference value determined experimentally (e.g., the correct density ρ₀).

At block 440, the electronic device 110 optimizes the analysis model with the target value of the target material regarding the first property. For example, the electronic device 110 may continue to fine-tune the analysis model with the target pressure value.

In some embodiments, after undergoing two rounds of fine tuning, the electronic device 110 may utilize the fine-tuned analysis model to determine a first value of the target material regarding the second property, i.e., a new density prediction.

In some embodiments, if the difference between the first value (e.g., the new density prediction) and the reference value (e.g., the correct density ρ₀) is less than or equal to the first threshold, the electronic device 110 determines the fine-tuned analysis model as the optimized analysis model.

Conversely, if the difference between the first value and the reference value is greater than the first threshold, the electronic device 110 may continue to fine-tune the model. Specifically, the electronic device 110 may determine a second association between the first property and the second property of the target material based on the first set of values, the second set of values, the preset value for the first property (e.g., P₁), and the second value for the second property (e.g., ρ₁). For example, the electronic device 110 may perform linear fitting using the two-wheel prediction result to improve the accuracy of the linear fitting.

Similarly, the electronic device 110 may determine, based on the reference value of the target material regarding the second property, a second target value of the target material regarding the first property using the second association, for example, a new target pressure value. Accordingly, the electronic device 110 may optimize the analysis model with a second target value (e.g., a new target pressure value) of the target material regarding the first property.

Based on the above manner, the embodiments of the present disclosure can optimize the analysis model by using limited experimental data.

### Knowledge Distillation

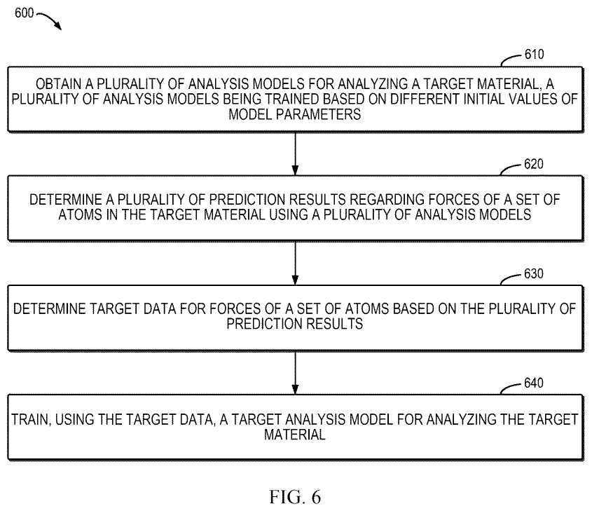

FIG. 6 illustrates a flowchart of an example process 600 of constructing a ligand generative model according to some embodiments of the present disclosure. Process 600 may be implemented at electronic device 110. The process 600 is described below with reference to FIG. 1.

At block 610, the electronic device 110 obtains a plurality of analysis models for analyzing a target material, a plurality of analysis models being trained based on different initial values of model parameters.

In some embodiments, the electronic device 110 may train the plurality of analysis models based on different random seeds.

At block 620, the electronic device 110 determines a plurality of prediction results regarding forces of a set of atoms in the target material using a plurality of analysis models.

At block 630, the electronic device 110 determines target data for forces of a set of atoms based on the plurality of prediction results.

Specifically, the electronic device 110 may determine an average value and/or a standard deviation of the plurality of prediction results, and may determine the target data for the forces of the set of atoms based on the average value and/or the standard deviation of the plurality of prediction results.

At block 640, the electronic device 110 train, using the target data, a target analysis model for analyzing the target material.

In some embodiments, the electronic device 110 may train the target analysis model such that: the target prediction result of the forces regarding the set of atoms by of the target analysis model is close to the average value of the plurality of prediction results; and/or the standard deviation prediction result of the forces regarding the set of atoms by of the target analysis model is close to the standard deviation of the plurality of prediction results.

Thus, by utilizing the mean and standard deviation, embodiments of the present disclosure may implement knowledge distillation such that a single model possesses the ability to approximate the overall prediction capability and uncertainty estimation of multiple model.

In some embodiments, considering that the standard deviation indicates the uncertainty of the plurality of models for the prediction result, if the standard deviation is greater than the fourth threshold, the electronic device 110 may further obtain reference data about the force of the target material, and may train the target analysis model based on the reference data.

For example, for a new sample, if the prediction standard deviation of the plurality of models for the sample is large, the analysis result uncertainty of the plurality of models is higher. Thus, the electronic device 110 may further obtain reference data about the sample to continue training the plurality of models. In some embodiments, such reference data may be generated, for example, based on a density functional theory DFT.

In some embodiments, the target analysis model to be trained may be, for example, a new model, or at least one analysis model of a plurality of analysis models trained based on different initial values of the model parameters.

### Experimental Results

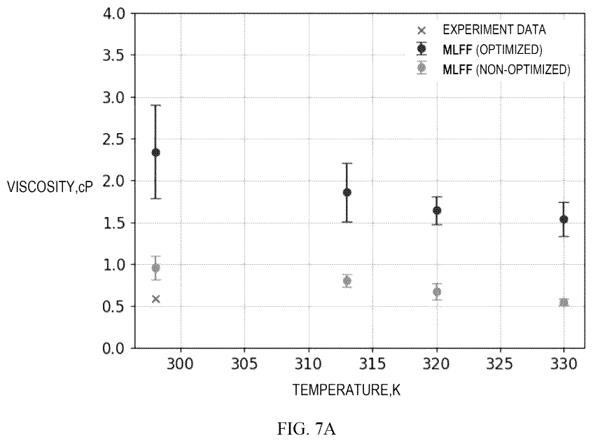

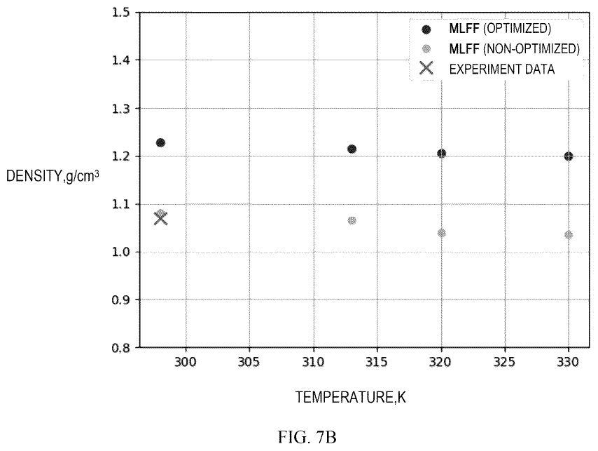

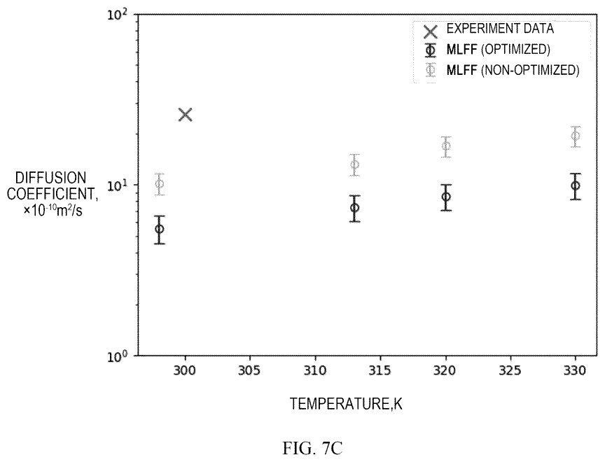

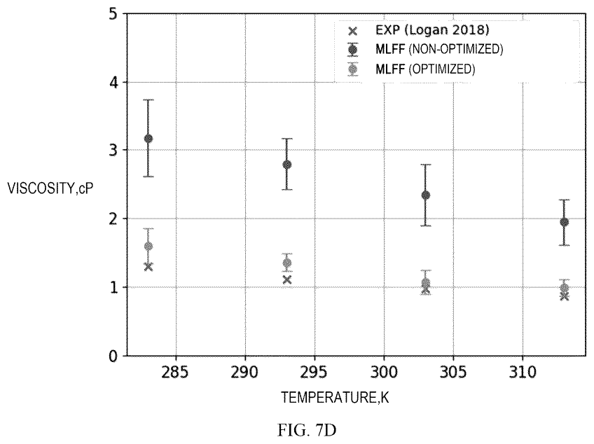

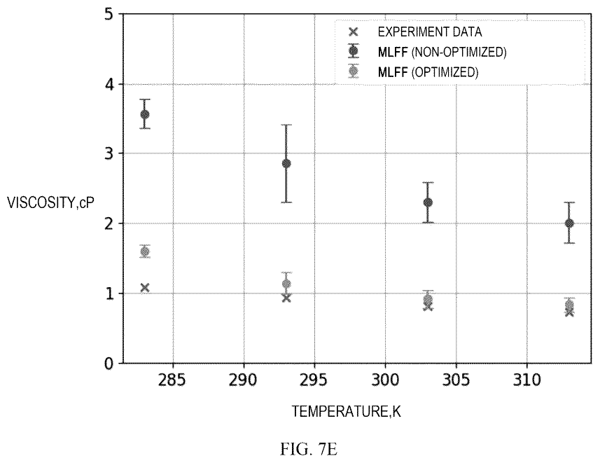

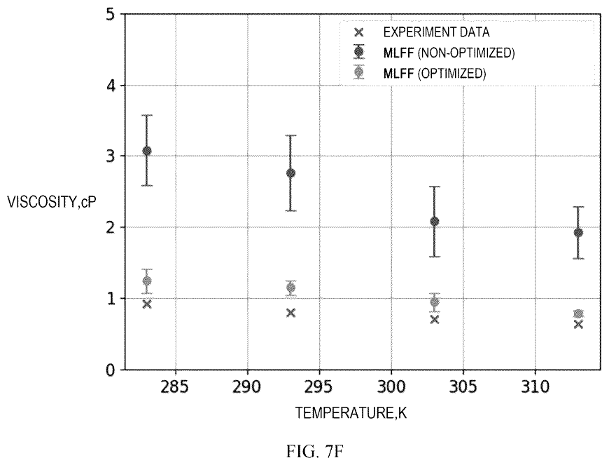

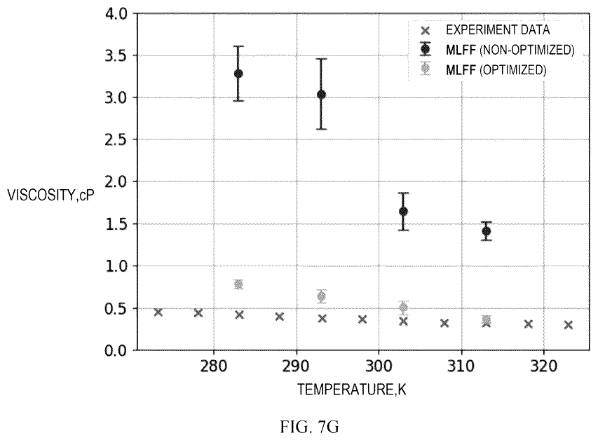

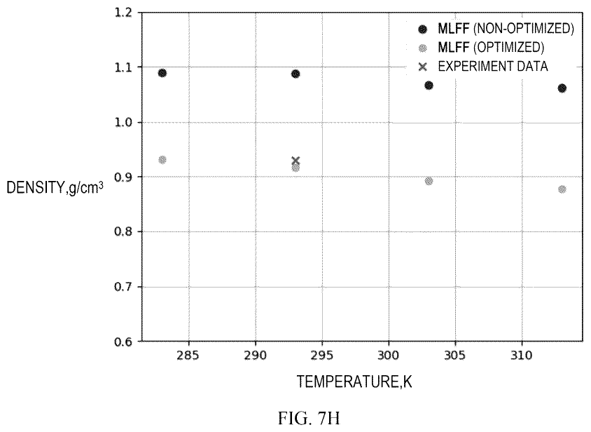

A comparison of analysis models (including optimized and unoptimized) and experimental data according to the present disclosure will be described below in conjunction with FIGS. 7A-7I. The DMC, EC, EMC, MA in the figures represent the name of Dimethyl carbamate, enthylene carbonate, and methyl acrylate solvent, respectively.

FIGS. 7A-7C show a comparison of viscosity, density, and diffusion coefficient of a DMC pure solvent. FIGS. 7A-7C present prediction results for the viscosity (FIG. 7A), density (FIG. 7B), and diffusion coefficient (FIG. 7C) for DMC as a neat solvent at different temperatures, optimized, and not optimized. Significant differences exist between the optimized results and the unoptimized results. Although model optimization depends only on the density of the DMC, it can be observed that the prediction accuracy for the viscosity and diffusion coefficients of the DMC is also significantly improved.

FIGS. 7D-7F show the viscosity prediction of the EC-EMC mixed solvent. FIGS. 7D-7F illustrate the results of predictions of viscosity by optimized and unoptimized model at different temperatures for different proportions of the two-element mixed solvent EC-EMC. FIG. 7D shows the weight ratio of EC-EMC to 30:70; FIG. 7E is 20:80; and FIG. 7F is 10:90. It can be clearly seen that in different mixing solvent ratios and temperature ranges, the optimized model can better match the experimental results, and this accuracy is not significant in the unoptimized model. Therefore, based on the model only optimizing the limited number of pure solvent physical property data, the solution of the present disclosure demonstrates excellent generalization ability in terms of mixed solvent and temperature range.

FIGS. 7G-7I show a model unseen solvent MA property prediction. FIGS. 7G-7I illustrate prediction predictive ability of the model on unseen solvent MA. This model for predicting MA characteristics was not optimized according to the experimental physical properties of MA. Here, optimization refers to over-optimization on a solvent similar to the MA structure. FIG. 7G shows the viscosity of MA, FIG. 7H is the density, and FIG. 7I is the diffusion coefficient. It can be seen that although the model has never been optimized on MA from unseen MA molecules, it has been able to master interactions between MA molecules and molecules by learning molecules similar to MA. This makes the model accurately prediction in terms of density, exhibiting excellent generalization ability in terms of viscosity and diffusion coefficients.

### EXAMPLE APPARATUS AND APPARATUS

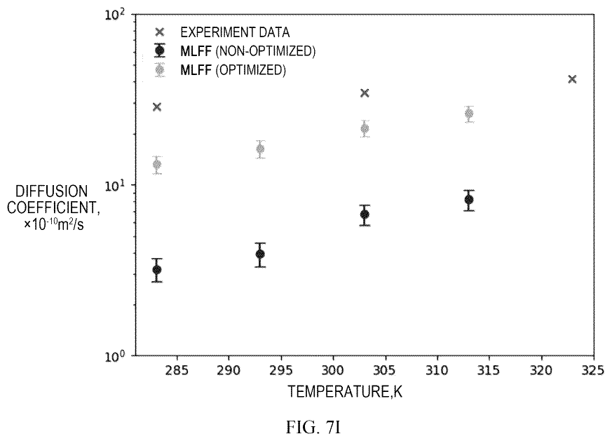

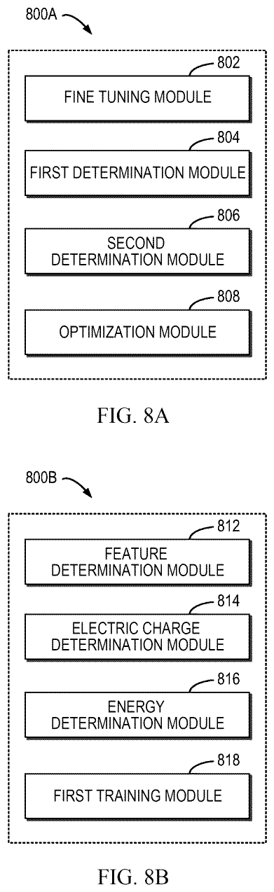

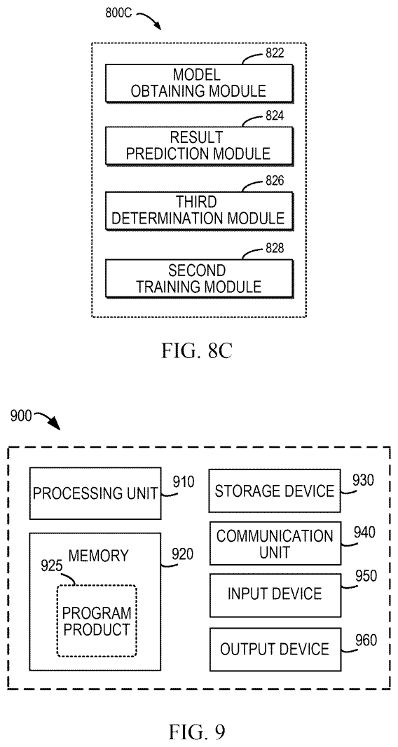

Embodiments of the present disclosure also provide a corresponding apparatus for implementing the above method or process. FIG. 8A illustrates a schematic structural block diagram of an apparatus 800A for optimizing an analysis model according to some embodiments of the present disclosure. The device 800A may be implemented or included in the electronic device 110. The various modules/components in the apparatus 800A may be implemented by hardware, software, firmware, or any combination thereof.

As shown in FIG. 8A, the apparatus 800A comprises: a fine-tuning model 802 configured to fine-tune an analysis model with a first set of values regarding a first property of a target material to determine a second set of values regarding a second property of the target material; a first determination model 804 configured to determine an association between the first property and the second property of the target material based on a first set of values and a second set of values; a second determination module 806 configured to determine a target value of the target material regarding the first property with the association based on a reference value of the target material regarding the second property, the reference value being determined based on an experiment on target material; and a optimization module 808 configured to optimize the analysis model with the target value of the target material regarding the first property.

In some embodiments, the fine-tuning module 802 is further configured to: fine-tune the analysis model with a first set of values regarding the first property to determine a set of intermediate models; and determine the second set of values of the target material regarding the second property using the set of intermediate models.

In some embodiments, the first determination module 804 is further configured to linearly fit at least the first set of values and the second set of values to determine the association between the first property and the second property of the target material.

In some embodiments, the optimization module 808 is further configured to: fine-tune the analysis model using the target value; determine a first value of the target material regarding the second property using the fine-tuned analysis model; and in response to a difference between the first value and the reference value being less than or equal to a first threshold, determine the fine-tuned analysis model as an optimized analysis model.

In some embodiments, the association is a first association, the target value is a first target value, and the optimization module 808 is further configured to: in response to a difference between the first value and the reference value being greater than the first threshold, determine a second association between the first property and the second property of the target material based on the first set of values, the second set of values, a preset value for the first property, and a second value for the second property, the second value corresponding to the preset value of the first property; determine a second target value of the target material regarding the first property using the second association based on the reference value of the target material regarding the second property; and optimize the analysis model using the second target value of the target material regarding the first property.

In some embodiments, the first set of values are determined based on the preset value and at least one variation of the first property.

In some embodiments, the second value is determined by the analysis model based on an NPT ensemble.

In some embodiments, the target material comprises a solution, the first property is a pressure of the solution, and the second property is a density of the solution.

In some embodiments, the target material comprises a solution, the first property is a dipole moment of the solution, and the second property is an electrical conductivity of the solution.

Embodiments of the present disclosure also provide a corresponding apparatus for implementing the above method or process. FIG. 8B shows a schematic structural block diagram of an apparatus 800B for training an analysis model according to some embodiments of the present disclosure. The device 800B may be implemented or included in the electronic device 110. The various modules/components in the apparatus 800B may be implemented by hardware, software, firmware, or any combination thereof.

As shown in FIG. 8B, the apparatus 800B comprises: a feature determination module 812 configured to determine a set of feature representations of a set of atoms in an electrolyte sample using an analysis model; an electric charge determination module 814 configured to determine electric charge information of the electrolyte sample using the analysis model based on the set of feature representations; an energy determination module 816 configured to determine energy information of the electrolyte sample with the analysis model based on the electric charge information and the set of feature representations; and a first training module 818 configured to train the analysis model based on the electric charge information and the energy information, wherein a loss function for training the analysis model comprises at least a first part and/or a second part, the first part being associated with a multipolar moment determined based on the electric charge information, the second part being associated with force information determined based on the energy information.

In some embodiments, the loss function further comprises a third part associated with the electric charge information and/or a dipole moment determined based on the electric charge information.

In some embodiments, the force information is a Wiener tensor determined based on the energy information.

In some embodiments, a value of the loss function is determined based on a comparison with reference data for the electrolyte sample, wherein the reference data is generated based on a density functional theory DFT.

In some embodiments, the feature determination module 812 is further configured for: determining a graph representation corresponding to the set of atoms in the electrolyte sample, wherein a plurality of nodes in the graph representation correspond to the set of atoms, an edge in the graph representation indicating that a distance between corresponding two atoms is less than a second threshold; and determining the set of feature representations of the set of atoms in the electrolyte sample by processing the graph representation with a graph neural network of the analysis model.

In some embodiments, the feature determination module 812 is further configured for: determining a first feature representation for a first atom of the set of atoms; determining at least one second atom based on a distance from the set of atoms to the first atom, wherein a distance from the at least one second atom to the first atom is less than a third threshold; updating the first feature representation of the first atom using the feature representation of the at least one second atom; and determining the set of feature representations of the set of atoms based on the updated first feature representation.

Embodiments of the present disclosure also provide a corresponding apparatus for implementing the above method or process. FIG. 8C shows a schematic structural block diagram of an apparatus 800C for training an analysis model according to some embodiments of the present disclosure. The device 800C may be implemented or included in the electronic device 110. The various modules/components in the apparatus 800C may be implemented by hardware, software, firmware, or any combination thereof.

As shown in FIG. 8C, the apparatus 800C comprises: a model obtaining module 822 configured to obtain a plurality of analysis models for analyzing a target material, a plurality of analysis models being trained based on different initial values of model parameters; a result prediction module 824 configured to determine a plurality of prediction results regarding forces of a set of atoms in the target material using a plurality of analysis models; a third determination module 826 configured to determine target data for forces of a set of atoms based on the plurality of prediction results; and second training module 828 configured to train, using the target data, a target analysis model for analyzing the target material.

In some embodiments, the third determining module 826 is further configured for: determining an average value and/or a standard deviation of the plurality of prediction results; and determining the target data for the force of the set of atoms based on the average value and/or the standard deviation of the plurality of prediction results.

In some embodiments, the second training module 828 is further configured for training the target analysis model such that: a target prediction result of the forces regarding the set of atoms by the target analysis model is close to the average value of the plurality of prediction results; and/or a standard deviation prediction result of the forces regarding the set of atoms by the target analysis model is close to the standard deviation of the plurality of prediction results.

In some embodiments, the apparatus 800C further includes a third training module configured for: in response to the standard deviation being greater than a fourth threshold, obtaining reference data of the forces of the target material, and training the target analysis model based on the reference data.

In some embodiments, the reference data is generated based on a density functional theory DFT.

In some embodiments, the target analysis model comprises at least one analysis model of the plurality of analysis models.

FIG. 9 illustrates a block diagram of an electronic device 900 in which one or more embodiments of the present disclosure may be implemented. It should be understood that the electronic device 900 illustrated in FIG. 9 is merely exemplary and should not constitute any limitation on the functionality and scope of the embodiments described herein. The electronic device 900 shown in FIG. 9 may be configured to implement the electronic device 110 in FIG. 1.

As shown in FIG. 9, the electronic device 900 is in the form of a general-purpose computing device. Components of the electronic device 900 may include, but are not limited to, one or more processors or processing units 910, a memory 920, a storage device 930, one or more communication units 940, one or more input devices 950, and one or more output devices 960. The processing unit 910 may be an actual or virtual processor and capable of performing various processes according to programs stored in the memory 920. In multiprocessor system, multiple processing units execute computer-executable instructions in parallel to improve parallel processing capabilities of electronic device 900.

Electronic device 900 typically includes a plurality of computer storage media. Such media may be any available media accessible to the electronic device 900, including, but not limited to, volatile and non-volatile media, removable and non-removable media. The memory 920 may be volatile memory (e.g., registers, caches, random access memory (RAM)), non-volatile memory (e.g., read-only memory (ROM), electrically erasable programmable read-only memory (EEPROM), flash memory), or some combination thereof. Storage device 930 may be a removable or non-removable medium and may include a machine-readable medium, such as a flash drive, magnetic disk, or any other medium, which may be capable of storing information and/or data (e.g., training data for training) and may be accessed within electronic device 900.

The electronic device 900 may further include additional removable/non-removable, volatile/non-volatile storage media. Although not shown in FIG. 9, a disk drive for reading or writing from a removable, nonvolatile magnetic disk (e.g., a “floppy disk”) and an optical disk drive for reading or writing from a removable, nonvolatile optical disk may be provided. In these cases, each drive may be connected to a bus (not shown) by one or more data media interface. The memory 920 may include a computer program product 925 having one or more program modules configured to perform various methods or actions of various embodiments of the present disclosure.

The communication unit 940 is configured to communicate with another electronic device through a communication medium. Additionally, the functionality of components of the electronic device 900 may be implemented in a single computing cluster or multiple computing machines capable of communicating over a communication connection. Thus, the electronic device 900 may operate in a networked environment using logical connections with one or more other servers, network personal computers (PCs), or another network Node.

The input device 950 may be one or more input devices, such as a mouse, a keyboard, a trackball, or the like. The output device 960 may be one or more output devices, such as a display, a speaker, a printer, or the like. The electronic device 900 may also communicate with one or more external devices (not shown) through the communication unit 940 as needed, external devices such as storage devices, display devices, etc., communicate with one or more devices that enable a user to interact with the electronic device 900, or communicate with any device (e.g., a network card, a modem, etc.) that enables the electronic device 900 to communicate with one or more other electronic devices. Such communication may be performed via an input/output (I/O) interface (not shown).

According to example embodiments of the present disclosure, there is provided a computer-readable storage medium having computer-executable instructions stored thereon, wherein the computer-executable instructions are executed by a processor to implement the method described above. According to an exemplary embodiment of the present disclosure, a computer program product is further provided, the computer program product being tangibly stored on a non-transient computer readable medium and comprising computer executable instructions, the computer executable instructions being executed by a processor to implement the method described above.

Aspects of the present disclosure are described herein with reference to flowcharts and/or block diagrams of methods, apparatuses, devices, and computer program products implemented in accordance with the present disclosure. It should be understood that each block of the flowchart and/or block diagram, and combinations of blocks in the flowcharts and/or block diagrams, may be implemented by computer readable program instructions.

These computer-readable program instructions may be provided to a processing unit of a general purpose computer, special purpose computer, or other programmable data processing apparatus to produce a machine, such that the instructions, when executed by a processing unit of a computer or other programmable data processing apparatus, produce means to implement the functions/acts specified in the flowchart and/or block diagram. These computer-readable program instructions may also be stored in a computer-readable storage medium that cause the computer, programmable data processing apparatus, and/or other devices to function in a particular manner, such that the computer-readable medium storing instructions includes an article of manufacture including instructions to implement aspects of the functions/acts specified in the flowchart and/or block diagram(s).

The computer-readable program instructions may be loaded onto a computer, other programmable data processing apparatus, or other apparatus, such that a series of operational steps are performed on a computer, other programmable data processing apparatus, or other apparatus to produce a computer-implemented process such that the instructions executed on a computer, other programmable data processing apparatus, or other apparatus implement the functions/acts specified in the flowchart and/or block diagram block or blocks.

The flowchart and block diagrams in the figures show architecture, functionality, and operation of possible implementations of systems, methods, and computer program products according to various implementations of the present disclosure. In this regard, each block in the flowchart or block diagram may represent a module, program segment, or portion of an instruction that includes one or more executable instructions for implementing the specified logical function. In some alternative implementations, the functions noted in the blocks may also occur in a different order than noted in the figures. For example, two consecutive blocks may actually be performed substantially in parallel, which may sometimes be performed in the reverse order, depending on the functionality involved. It is also noted that each block in the block diagrams and/or flowchart, as well as combinations of blocks in the block diagrams and/or flowchart, may be implemented with a dedicated hardware-based system that performs the specified functions or actions, or may be implemented in a combination of dedicated hardware and computer instructions.

Various implementations of the present disclosure have been described above, which are exemplary, not exhaustive, and are not limited to the implementations disclosed. Many modifications and variations will be apparent to those of ordinary skill in the art without departing from the scope and spirit of the various implementations illustrated. The selection of the terms used herein is intended to best explain the principles of the implementations, the practical application, or improvements to the technology in the marketplace, or to enable others of ordinary skill in the art to understand the various embodiments disclosed herein.

I/We claim:

1. A method for optimizing an analysis model, comprising:
fine-tuning an analysis model with a first set of values regarding a first property of a target material to determine a second set of values regarding a second property of the target material;
determining an association between the first property and the second property of the target material based on a first set of values and a second set of values;
determining a target value of the target material regarding the first property with the association based on a reference value of the target material regarding the second property, the reference value being determined based on an experiment on target material; and
optimizing the analysis model with the target value of the target material regarding the first property.

2. The method of claim 1, wherein fine-tuning an analysis model with a first set of values regarding a first property of a target material comprises:
fine-tuning the analysis model with a first set of values regarding the first property to determine a set of intermediate models; and
determining the second set of values of the target material regarding the second property using the set of intermediate models.

3. The method of claim 1, wherein determining an association between the first property and the second property of the target material comprises:
linearly fitting at least the first set of values and the second set of values to determine the association between the first property and the second property of the target material.

4. The method of claim 1, wherein optimizing the analysis model with the target value of the target material regarding the first property comprises:
fine-tuning the analysis model using the target value;
determining a first value of the target material regarding the second property using the fine-tuned analysis model; and
in response to a difference between the first value and the reference value being less than or equal to a first threshold, determining the fine-tuned analysis model as an optimized analysis model.

5. The method of claim 4, wherein the association is a first association, the target value is a first target value, and optimizing the analysis model with the target value of the target material regarding the first property further comprises:
in response to a difference between the first value and the reference value being greater than the first threshold, determining a second association between the first property and the second property of the target material based on the first set of values, the second set of values, a preset value for the first property, and a second value for the second property, the second value corresponding to the preset value of the first property;
determining a second target value of the target material regarding the first property using the second association based on the reference value of the target material regarding the second property; and
optimizing the analysis model using the second target value of the target material regarding the first property.

6. The method of claim 5, wherein the first set of values are determined based on the preset value and at least one variation of the first property.

7. The method of claim 6, wherein the second value is determined by the analysis model based on an NPT ensemble.

8. The method of claim 1, wherein the target material comprises a solution, the first property is a pressure of the solution, and the second property is a density of the solution.

9. The method of claim 1, wherein the target material comprises a solution, the first property is a dipole moment of the solution, and the second property is an electrical conductivity of the solution.

10. A method for training an analysis model, comprising:
determining a set of feature representations of a set of atoms in an electrolyte sample using an analysis model;
determining electric charge information of the electrolyte sample using the analysis model based on the set of feature representations;
determining energy information of the electrolyte sample with the analysis model based on the electric charge information and the set of feature representations; and
training the analysis model based on the electric charge information and the energy information, wherein a loss function for training the analysis model comprises at least a first part and/or a second part, the first part being associated with a multipolar moment determined based on the electric charge information, the second part being associated with force information determined based on the energy information.

11. The method of claim 10, wherein the loss function further comprises a third part associated with the electric charge information and/or a dipole moment determined based on the electric charge information.

12. The method of claim 10, wherein the force information is a Wiener tensor determined based on the energy information.

13. The method of claim 10, wherein a value of the loss function is determined based on a comparison with reference data for the electrolyte sample, wherein the reference data is generated based on a density functional theory DFT.

14. The method of claim 10, wherein determining a set of feature representations of a set of atoms in an electrolyte sample using an analysis model comprises:
determining a graph representation corresponding to the set of atoms in the electrolyte sample, wherein a plurality of nodes in the graph representation correspond to the set of atoms, an edge in the graph representation indicating that a distance between corresponding two atoms is less than a second threshold; and
determining the set of feature representations of the set of atoms in the electrolyte sample by processing the graph representation with a graph neural network of the analysis model.

15. The method of claim 14, wherein determining the set of feature representations of the set of atoms in the electrolyte sample by processing the graph representation with a graph neural network of the analysis model comprises:
determining a first feature representation for a first atom of the set of atoms;
determining at least one second atom based on a distance from the set of atoms to the first atom, wherein a distance from the at least one second atom to the first atom is less than a third threshold;
updating the first feature representation of the first atom using the feature representation of the at least one second atom; and
determining the set of feature representations of the set of atoms based on the updated first feature representation.

16. A method for training an analysis model, comprising:
obtaining a plurality of analysis models for analyzing a target material, a plurality of analysis models being trained based on different initial values of model parameters;
determining a plurality of prediction results regarding forces of a set of atoms in the target material using a plurality of analysis models;
determining target data for forces of a set of atoms based on the plurality of prediction results; and
training, using the target data, a target analysis model for analyzing the target material.

17. The method of claim 16, wherein determining target data for forces of a set of atoms based on the plurality of prediction results comprises:
determining an average value and/or a standard deviation of the plurality of prediction results; and
determining the target data for the force of the set of atoms based on the average value and/or the standard deviation of the plurality of prediction results.

18. The method of claim 17, wherein training, using the target data, a target analysis model for analyzing the target material comprises:
training the target analysis model such that:
a target prediction result of the forces regarding the set of atoms by the target analysis model is close to the average value of the plurality of prediction results; and/or
a standard deviation prediction result of the forces regarding the set of atoms by the target analysis model is close to the standard deviation of the plurality of prediction results.

19. The method of claim 17, further comprising:
in response to the standard deviation being greater than a fourth threshold, obtaining reference data of the forces of the target material, and
training the target analysis model based on the reference data.

20. The method of claim 19, wherein the reference data is generated based on a density functional theory DFT.

21. The method of claim 16, wherein the target analysis model comprises at least one analysis model of the plurality of analysis models.

22. An electronic device comprising:
at least one processing unit; and
at least one memory coupled to the at least one processing unit and storing instructions for execution by the at least one processing unit, the instructions, when executed by the at least one processing unit, causing the electronic device to perform actions comprising:
fine-tuning an analysis model with a first set of values regarding a first property of a target material to determine a second set of values regarding a second property of the target material;
determining an association between the first property and the second property of the target material based on a first set of values and a second set of values;
determining a target value of the target material regarding the first property with the association based on a reference value of the target material regarding the second property, the reference value being determined based on an experiment on target material; and
optimizing the analysis model with the target value of the target material regarding the first property.

23. An electronic device comprising:
at least one processing unit; and
at least one memory coupled to the at least one processing unit and storing instructions for execution by the at least one processing unit, the instructions, when executed by the at least one processing unit, causing the electronic device to perform actions comprising:
determining a set of feature representations of a set of atoms in an electrolyte sample using an analysis model;
determining electric charge information of the electrolyte sample using the analysis model based on the set of feature representations;
determining energy information of the electrolyte sample with the analysis model based on the electric charge information and the set of feature representations; and
training the analysis model based on the electric charge information and the energy information, wherein a loss function for training the analysis model comprises at least a first part and/or a second part, the first part being associated with a multipolar moment determined based on the electric charge information, the second part being associated with force information determined based on the energy information.

24. An electronic device comprising:
at least one processing unit; and
at least one memory coupled to the at least one processing unit and storing instructions for execution by the at least one processing unit, the instructions, when executed by the at least one processing unit, causing the electronic device to perform actions comprising:
obtaining a plurality of analysis models for analyzing a target material, a plurality of analysis models being trained based on different initial values of model parameters;
determining a plurality of prediction results regarding forces of a set of atoms in the target material using a plurality of analysis models;
determining target data for forces of a set of atoms based on the plurality of prediction results; and
training, using the target data, a target analysis model for analyzing the target material.
